(19) United States
(12) Patent Application Publication
(10) Pub. No.: US 2008/0027464 A1
(43) Pub. Date: Jan. 31, 2008

(54) SYSTEMS AND METHODS FOR PERFORMING MINIMALLY INVASIVE SURGICAL OPERATIONS

(76) Inventors: Frederic H. Moll, Woodside, CA (US); Daniel T. Wallace, Burlingame, CA (US); Gregory J. Stahler, San Jose, CA (US); Christopher R. Carlson, Menlo Park, CA (US); Frederico Barbagli, San Francisco, CA (US)

Correspondence Address: VISTA IP LAW GROUP LLP, 12930 Saratoga Avenue, Suite D-2, Saratoga, CA, 95070, US

(21) Appl. No.: 11/829,076

(22) Filed: Jul. 26, 2007

Related U.S. Application Data
(60) Provisional application No. 60/833,624, filed on Jul. 26, 2006.

(51) Int. Cl. A61B 19/00

(52) U.S. Cl. 606/130

(57) ABSTRACT

A robotic surgical system (100) includes an instrument driver (106) that is mounted on an operation table (104), and an instrument assembly (108) is operatively coupled to the instrument driver (106), wherein the instrument assembly (108) includes a flexible guide instrument and a component instrument carried in a lumen of the guide instrument, the component instrument including a light source, camera and laser energy fiber.

### RELATED APPLICATION DATA

The present application claims the benefit under 35 U.S.C. § 119 to U.S. Provisional Patent Application Ser. No. 60/833,624, filed on Jul. 26, 2006. The foregoing application is incorporated by reference into the present application in its entirety for all purposes.

### FIELD OF INVENTION

The invention relates generally to robotically controlled systems, such as telerobotic surgical systems, and more particularly to robotic catheter systems for performing minimally invasive diagnostic and therapeutic procedures.

### BACKGROUND

Robotic diagnostic and interventional systems and devices are well suited for use in performing minimally invasive medical procedures, as opposed to conventional techniques wherein a patient's body cavity is open to permit the surgeon's hands access to the internal organs. There is a need for highly controllable yet minimally sized systems to facilitate imaging, diagnosis, and treatment of tissues which may lie deeply and/or concealed within the body cavity of a patient, and which may be accessed through natural body orifices or percutaneous incisions and using naturally-occurring pathways such as blood vessels or other bodily lumens.

### SUMMARY OF THE INVENTION

In accordance with various embodiments of the present invention, a robotic surgical system for performing minimally invasive surgical procedures includes components of an instrument assembly that are configured to be navigated through tortuous natural body pathways to tissue structures inside a patient for performing diagnostic and/or interventional operations. In one embodiment, the robotic surgical system includes an instrument driver that is mounted on an operation table in sufficiently close proximity where a patient is located. An instrument assembly is operatively coupled to the instrument driver, wherein the instrument assembly includes components that are configured to penetrate through the skin of the patient either by way of a natural body orifice or a percutaneous incision. The components of the instrument assembly are navigated through tortuous natural body pathways to one or more target sites for performing minimally invasive surgical operations on tissues inside the patient. An operator control station is located remotely from the operation table such that the operator is at some distance away from the operation table and away from radiation sources that may be used in connection with the minimally invasive surgical procedures. The operator control station is connected to the instrument driver by a wire connection or a wireless link. The operator control station includes input, display, and monitor systems and devices for an operator to monitor the components of the instrument assembly and provide the necessary input to navigate those components for performing the minimally invasive operations inside the patient on the operation table. The operator control station also includes an electronics rack in which system circuitry comprising of system software, hardware, firmware, and combinations thereof that are configured to store, process, execute, etc. the operator input and operate, control, etc. the hardware, software, firmware and combinations thereof at the instrument driver, such that the instrument driver may properly execute the control mechanisms necessary for maneuvering and navigating components of the instrument assembly for performing minimally invasive operations on the tissues inside the patient who is lying on the operation table.

In accordance with various embodiments of the present invention, a method for performing minimally invasive surgical procedure using a robotic surgical system with components that are configured to be navigated through tortuous natural body pathways to tissue structures inside a patient for performing diagnostic and/or interventional operations is provided. The method includes penetrating the skin of a patient who is lying on an operation table with one or more components of an instrument assembly, wherein the instrument assembly is a subsystem of the robotic surgical system. The instrument assembly is operatively coupled to an instrument driver, wherein the instrument driver is mounted on the operation table. The instrument driver is connected to an operator control station; the connection may be accomplished by a wire link or wireless link. The operator control station includes system hardware, software, firmware, and combinations thereof that are configured to store, process, display, and execute input, output, etc. for the operation of the robotic catheter system. The method also includes navigating components of the instrument assembly through tortuous natural body pathways to one or more target sites in the body of the patient. The method further includes performing surgical procedures at the one or more target sites in the body of the patient using one or more components of the instrument assembly.

Other aspects and advantages of the present invention will become apparent from the following description, taken in conjunction with the accompanying drawings, illustrating by way of examples the principles of the invention.

### BRIEF DESCRIPTION OF THE DRAWINGS

The present invention will be readily understood by the following detailed description, taken in conjunction with accompanying drawings, illustrating by way of examples the principles of the invention. The drawings illustrate the design and utility of preferred embodiments of the present invention, in which like elements are referred to by like reference symbols or numerals. The objects and elements in the drawings are not necessarily drawn to scale, proportion, or precise positional relationships; instead emphasis is focused on illustrating the principles of the invention.

FIG. 1 illustrates one embodiment of a robotic surgical system.

FIG. 2 illustrates another embodiment of a robotic surgical system.

FIG. 3 illustrates one embodiment of a robotic surgical system being used to perform diagnostic and/or interventional operations on a patient.

FIG. 4 illustrates an instrument assembly with a lithotripsy laser fiber for performing extracorporeal shock wave lithotripsy procedures.

FIG. 5 illustrates an instrument assembly with a grasper including an energy source configured for performing lithotripsy procedures.

FIG. 6 illustrates an instrument assembly with a basket tool including an energy source configured for performing lithotripsy procedures.

FIG. 7 illustrates an expandable grasping tool assembly including an energy source.

FIG. 8 illustrates a bipolar electrode grasper assembly.

FIG. 9 illustrates an instrument assembly configured with basket arms.

FIG. 10 illustrates an instrument assembly including a lithotripsy fiber and image capture device.

FIG. 11 illustrates an instrument assembly including a grasping tool.

FIG. 12 illustrates an instrument assembly including a basket tool.

FIG. 13 and FIG. 14 illustrate an operation of an instrument assembly with a basket tool.

FIG. 15 illustrates an instrument assembly including a basket arm capture device and image capture device.

FIG. 16 illustrates an instrument assembly including a balloon apparatus.

FIG. 17 illustrates an instrument assembly including another balloon apparatus.

FIG. 18 illustrates an instrument assembly including yet another balloon apparatus.

FIG. 19 through FIG. 21 illustrate an instrument assembly including an inflatable balloon cuff.

FIG. 22 through FIG. 24 illustrate an instrument assembly including a flexible balloon cuff.

FIG. 25 and FIG. 27 illustrate an instrument assembly including image capture apparatuses.

FIG. 26 through FIG. 29 illustrate detailed views of the image capture assembly.

FIG. 29 illustrates a cross sectional view of a tubular structure for housing the image capture device assembly.

FIG. 30 through FIG. 33 illustrate variations of embodiments of image capture assembly.

FIG. 34 illustrates a steerable instrument assembly.

FIG. 35 illustrates another steerable instrument assembly.

FIG. 36 and FIG. 37 illustrate yet another steerable instrument assembly.

### DETAILED DESCRIPTION OF THE ILLUSTRATED EMBODIMENTS

Reference will now be made in detail to the preferred embodiments of the present invention, examples of which are illustrated in the accompanying drawings. While the invention will be described in conjunction with the preferred embodiments, it will be understood that they are not intended to limit the invention to these embodiments. On the contrary, the invention is intended to cover modifications, alternatives, and equivalents that may be included within the spirit and scope of the invention as defined by the appended claims. Furthermore, in the following detailed description of the embodiments, numerous specific details are set forth in to order to provide a thorough understanding of the present invention. However, it will be readily apparent to one skilled in the art that the present invention may be practiced without these specific details. In other instances, well-known methods, procedures, and components have not been described in detail so as not to unnecessarily obscure aspects of the present invention.

Standard surgical procedures typically involve using a scalpel to create an opening of sufficient size to enable a surgical team to gain access to an area in the body of a patient for the surgical team to diagnose and treat one or more target sites. When possible, minimally invasive surgical procedures may be used instead of standard surgical procedures to minimize physical trauma to the patient and reduce recovery time for the patient to recuperate from the surgical procedures. Minimally invasive surgical procedures typically require using extension tools (e.g., catheters, etc.) to approach and address the target site through natural pathways (e.g., blood vessels, gastrointestinal tract, etc.) from a remote location either through a natural body orifice or a percutaneous incision. As can be appreciated, the surgeon may have limited information or feedback (e.g., visual, tactile, etc.) to accurately navigate the extension tools, such as one or more catheters, and place the working portions of the extension tools at precise locations to perform the necessary diagnostic and/or interventional procedures. Even with such potential limitations, minimally invasive surgical procedures may be more effective and beneficial for treating the patient, instead of standard open surgery.

Minimally invasive diagnostic and interventional operations may require the surgeon to remotely approach and address the operation or target site by using extension tools. The surgeon usually approaches the target site through either a natural body orifice or a small percutaneous incision in the body of the patient. In some situations, the surgeon may use multiple extension tools and approach the target site through both a natural body orifice as well as a small percutaneous incision in the body of the patient. Typically, the natural body orifice or small incision is located at some distance away from the target site. Extension tools (e.g., various types of catheters and surgical instruments) enter the body through one or more natural body orifices or small percutaneous incisions, and the extension tools are guided, navigated, manipulated, maneuvered, and advanced toward the target site typically by way of natural body pathways (e.g., blood vessels, esophagus, trachea, small intestine, large intestine, urethra, etc.). The extension tools might include one or more catheters as well as other surgical tools or instruments. The catheters may be manually controlled catheters or robotically operated catheters. In most situations, the surgeon has limited visual and tactile information to discern the location of the catheters and surgical instruments relative to the target site and/or other organs in the patient.

For example, in the treatment of cardiac arrhythmias such as atrial fibrillation (AF), cardiac ablation therapy is applied to the left atrium of the heart to restore normal heart function. For this operation, one or more catheters (e.g., sheath catheter, guide catheter, ablation catheter, endoscopic catheter, intracardiac echocardiography catheter, etc.) may be inserted through one or more natural orifices or one or more percutaneous incisions at the femoral vein near the thigh or pelvic region of the patient, which is located at some distance away from the operation or target site. In this example, the operation or target site for performing cardiac ablation is in the left atrium of the heart. Catheters may be guided (e.g., by a guide wire, a sheath, etc.), manipulated, maneuvered, and advanced toward the target site by way of the femoral vein to the inferior vena cava into the right atrium of the heart and through the interatrial septum to the left atrium of the heart. The catheters may be used separately or in combination of multiple catheters. Currently, the surgeon has limited visual and tactile information to assist him or her with maneuvering and controlling the catheters (separately or in combination). In particular, because of limited information and/or feedback, it is especially difficult for the surgeon to maneuver and control one or more distal portions of the catheters to perform cardiac ablation at precise locations or spots on the surface or wall of the left atrium of the heart. As will be explained below, embodiments of the present invention provide improved systems and methods that would facilitate imaging, diagnosis, address, and treatment of tissues which may lie deeply and/or concealed under other tissues or organs within the body cavity of a patient. With embodiments of the present invention, the surgeon may be able to position the catheter more precisely and accurately to address the operation or target sites. For example, with the improved imaging capability, the surgeon may be able to apply cardiac ablation at the desired locations or spots on the surface or wall of the left atrium of the heart in a more precise and accurate manner to address cardiac arrhythmias such as atrial fibrillation.

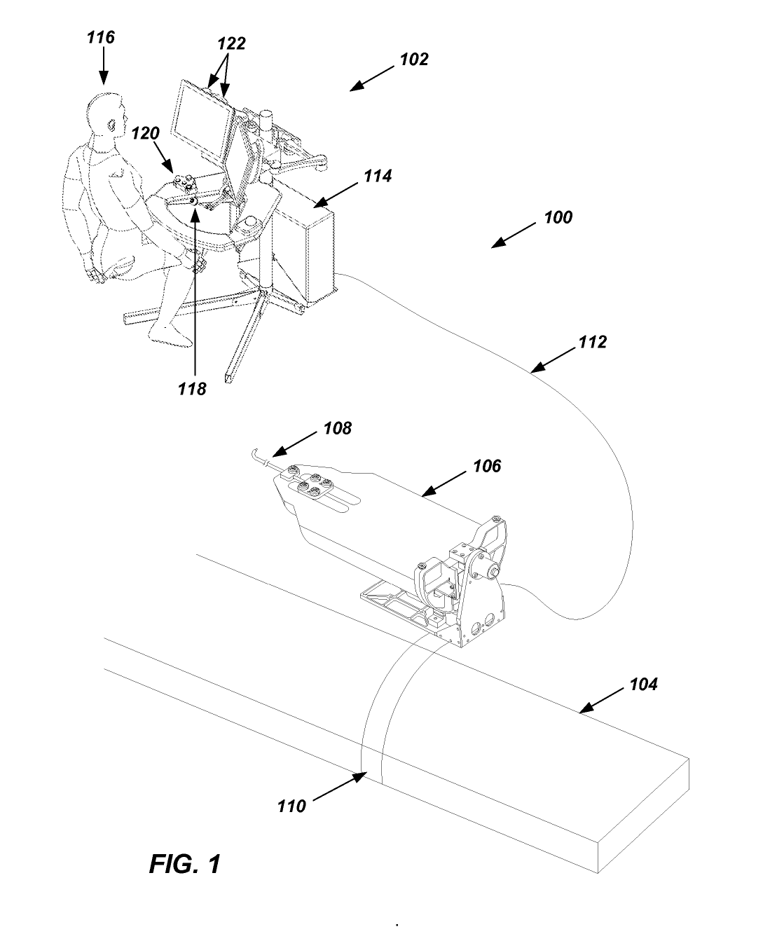

FIG. 1 illustrates one embodiment of a robotic surgical system (100), e.g., the Sensei™ Robotic Catheter System from Hansen Medical, Inc. in Mountain View, Calif., U.S.A., an operator control station (102) located remotely from an operating table (104) to which an instrument driver (106) and instrument (108), e.g., the Artisan™ Control Catheter also from Hansen Medical, Inc. in Mountain View, Calif., U.S.A., are supported by an instrument driver mounting brace (110). A wired connection (112) transfers signals between an electronics rack (114) at the operator control station (102) and instrument driver (106). The electronics rack (114) includes system hardware and software that substantially operate and perform the many functions of the robotic surgical system (100). The instrument driver mounting brace (110) is a substantially arcuate-shaped structural member configured to position the instrument driver (106) above a patient (not shown) lying on the operating table (104). The wired connection (112) may transmit manipulation and control commands from an operator or surgeon (116) who is working at the operator control station (102) to the instrument driver (106) to operate the instrument (108) to perform minimally invasive operations on the patient lying on the operating table (104). The surgeon (116) may provide manipulation and control commands using a master input device (MID) (118). In addition, the surgeon may provide inputs, commands, etc. by using one or more keyboards (120), trackball, mouse, etc. The wired connection (112) may also transmit information (e.g., visual views, tactile or force information, position, orientation, shape, localization, electrocardiogram, map, model, etc.) from the instrument (108), the patient, and monitors (not shown in this figure) to the electronics rack (114) for providing the necessary information or feedback to the operator or surgeon (116) to facilitate monitoring of the instrument (108), the patient, and one or more target sites for performing precise manipulation and control of the instrument (108) during the minimally invasive surgical procedure. The wired connection (112) may be a hard wire connection, such as an electrical wire configured to transmit electrical signals (e.g., digital signals, analog signals, etc.), an optical fiber configured to transmit optical signals, a wireless link configured to transmit various types of signals (e.g., RF signals, microwave signals, etc.), etc., or any combinations of electrical wire, optical fiber, wireless link, etc. The information or feedback may be displayed on one or more monitors (122) at the operator control station (102).

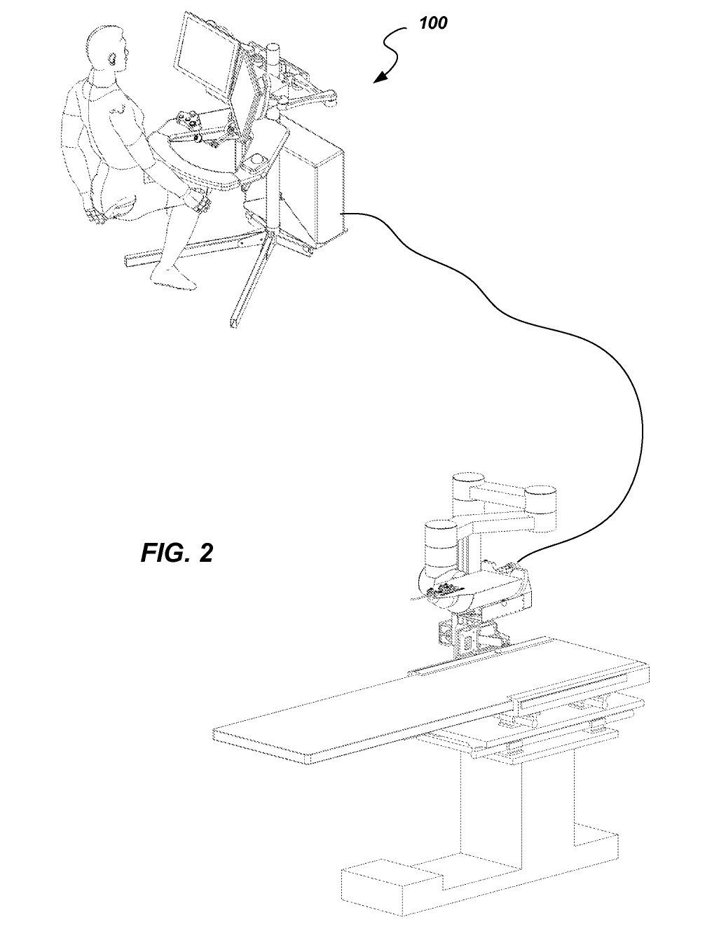

FIG. 2 illustrates another embodiment of a robotic surgical system (100). For more detailed discussions of the robotic surgical systems, please refer to U.S. Provisional Patent Application No. 60/644,505, filed on Jan. 13, 2005; U.S. patent application Ser. No. 11/481,433, filed on Jul. 3, 2006; and U.S. patent application Ser. No. 11/637,951, filed on Dec. 11, 2006; and they are incorporated herein by reference in their entirety.

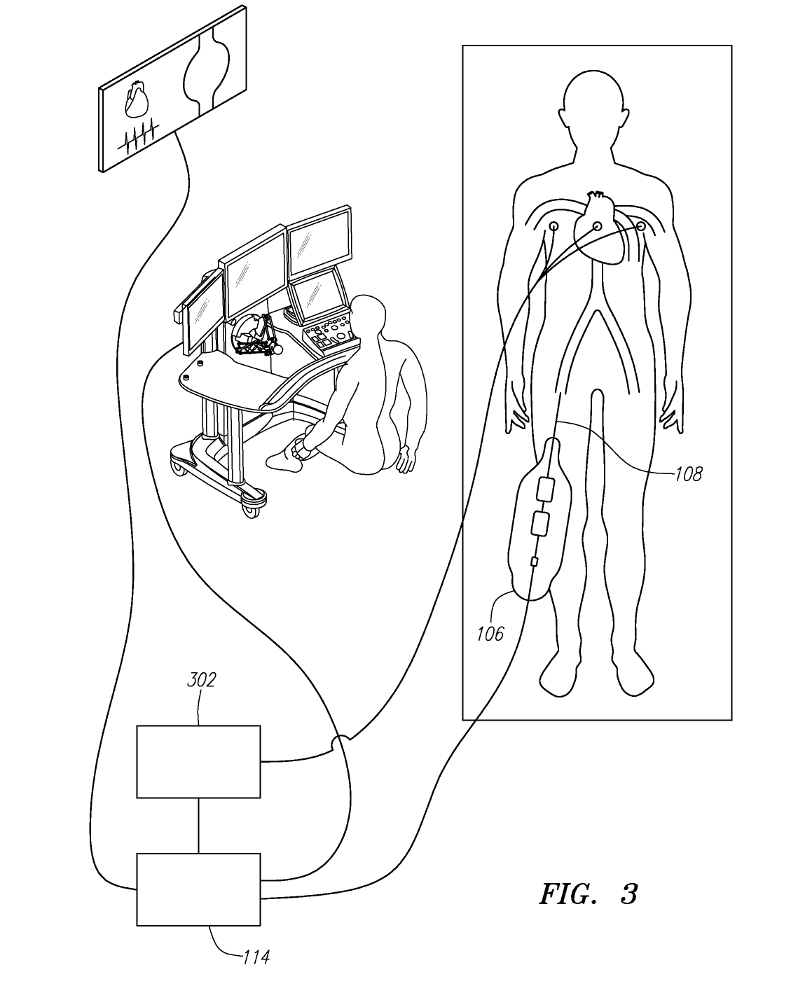

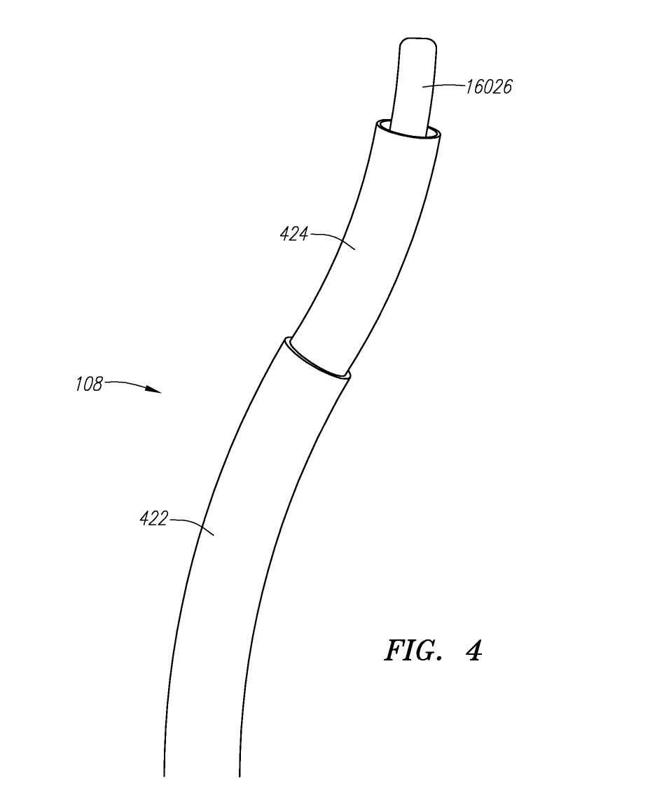

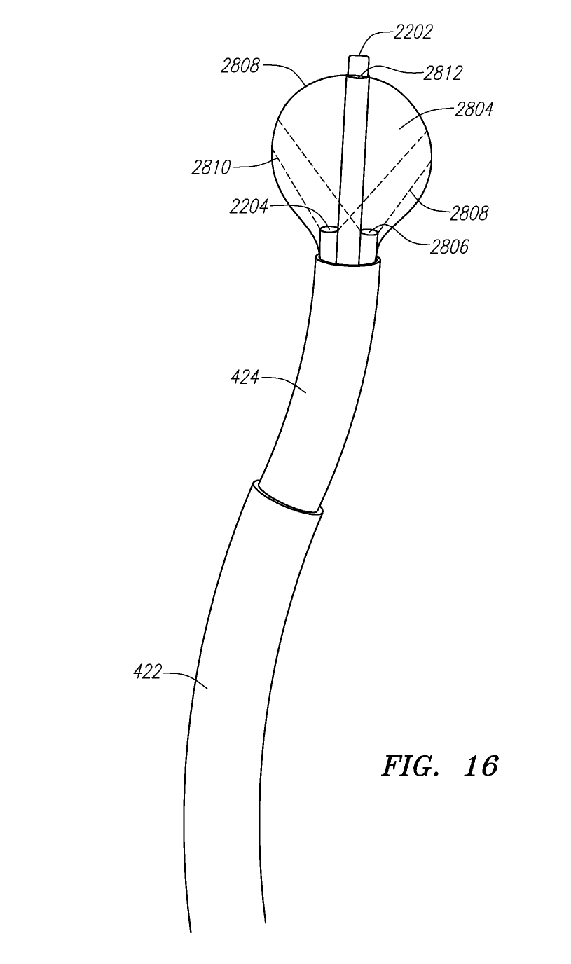

FIG. 3 illustrates one embodiment of a robotic surgical system (100) configured to perform minimally invasive surgery using one or more instruments (108). For example, the instrument (108) may be a sheath catheter, guide catheter, ablation catheter, endoscopic catheter, intracardiac echocardiography catheter, etc., or any combination thereof. In addition, surgical instruments or tools may be attached to any one or combination of the catheters. In one embodiment, the instrument (108) may be a catheter system that includes a sheath catheter, guide catheter, a surgical catheter, and/or surgical instrument, such as the Artisan™ Control Catheter available from Hansen Medical, Inc. at Mountain View, Calif., U.S.A. The instrument (108) also includes all the control mechanism to operate its various components, e.g., sheath catheter, guide catheter, a surgical catheter, and/or surgical instrument the robotic surgical system (100) including the control station (102), instrument driver (106), instrument (108), and the wired connection (112) may be used to treat diseases, maladies, or conditions in the tissues or organs of the digestive system, colon, urinary system, reproductive system, etc. For example, the robotic surgical system (100) may be used to perform Extracorporeal Shock Wave Lithotripsy (ESVL). FIG. 4 illustrates one embodiment of instrument (108) configured to perform ESWL. As illustrated in FIG. 16, instrument (108) may include a sheath catheter (422), a guide catheter (424), and a lithotripsy laser fiber (16026). Analogous to the discussion above, components or subsystems of the instrument (108) may be guided, manipulated, or navigated to the kidney to perform various operations. For example, subsystems of the instrument (108) may be guided, manipulated, or navigated to the kidney to remove kidney stones as oppose to similar components or subsystems of embodiments of the instrument (108), e.g., an ablation catheter, being guided, manipulated, or navigated to the left atrium of the heart to performing cardiac ablation to address cardiac arrhythmias. The lithotripsy laser fiber (16026) may include a quartz fiber coupled, connected to, or associated with a laser, such as a Holmium YAG laser, to apply energy to objects such as kidney stones, etc. In one configuration, the laser source may be positioned and interfaced with the fiber (16026) proximally, as in a typical lithotripsy configuration, with the exception that in the subject embodiment, the fiber (1602) is positioned down the working lumen of one or more robotic catheters (e.g., sheath catheter (422) and guide catheter (424)). All the necessary power source and control mechanisms including hardware and software to operate the laser may be located in the electronics rack (114) near the operator control station (102) of the robotic surgical system (100)

Since the distal tip of the lithotripsy fiber (16026) is configured to deliver energy to a target object, such as a kidney stone, the distal tip may be more generically described as an energy source. Indeed, in other embodiments, other energy sources, besides a laser, may be used to affect tissue. For example, in other embodiments, the energy source may be comprised of an RF electrode, an ultrasonic transducer, such as a high-frequency ultrasonic transducer, or other radiative, conductive, ablative, or convective energy source.

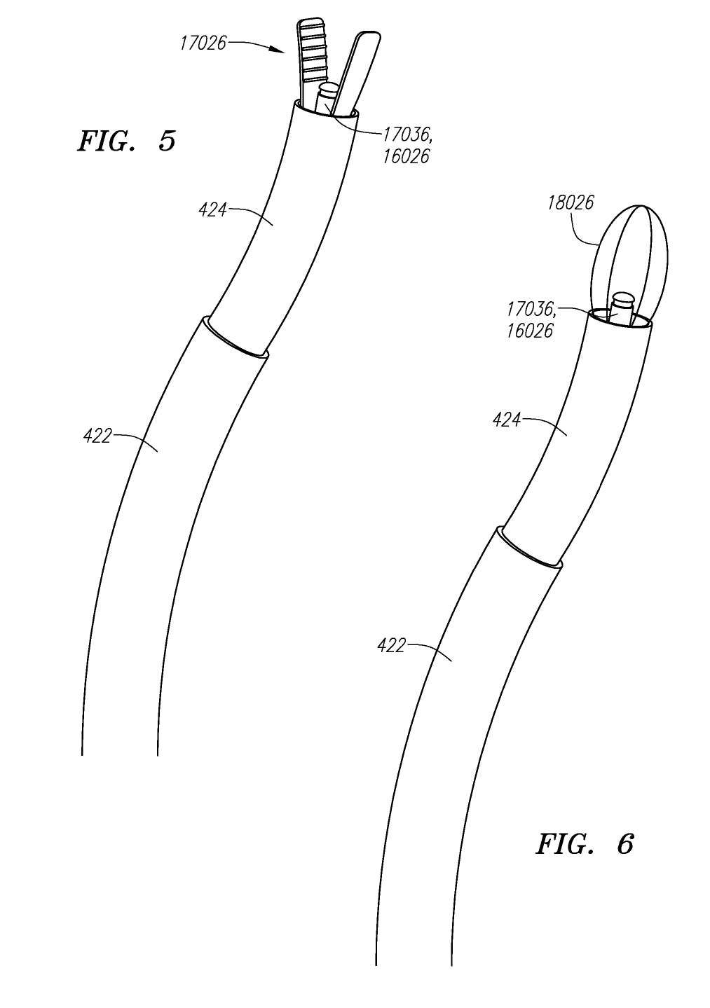

As may appreciated, the components or subsystems of instrument (108) may be configured with numerous different instruments or tool for performing various minimally invasive operations. For example, FIG. 5 depicts a guide instrument (424) operatively coupled to a grasper (17026) fitted with an energy source (17036), such as a lithotripsy laser fiber (16026) in a configuration wherein an object, such as a kidney stone, grasped within the clutches of the grasper (17026), may also be ablated, destroyed, fragmented, etc, by applied energy from the source (17036), which is positioned to terminate approximately at the apex of the grasper (17026) which it is likely to be adjacent to captured objects.

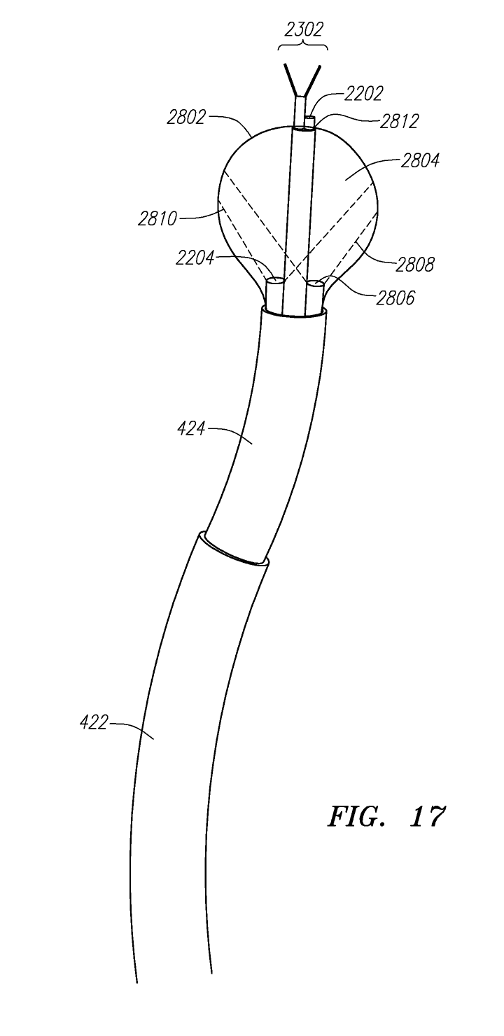

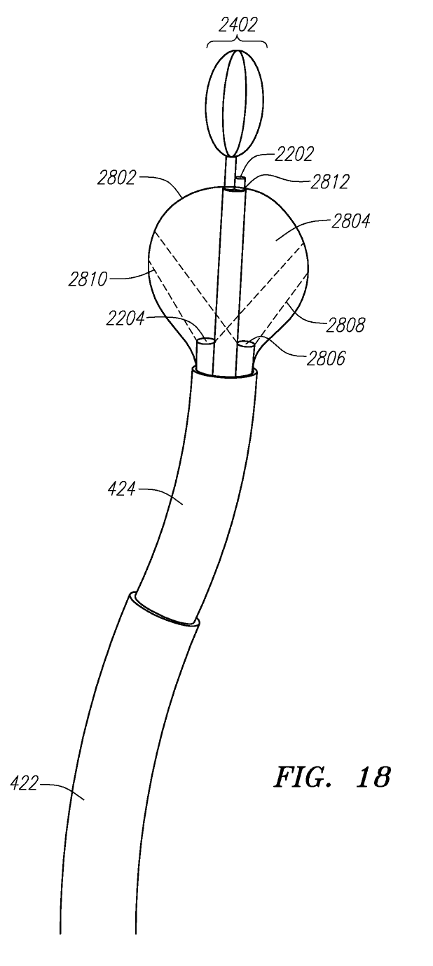

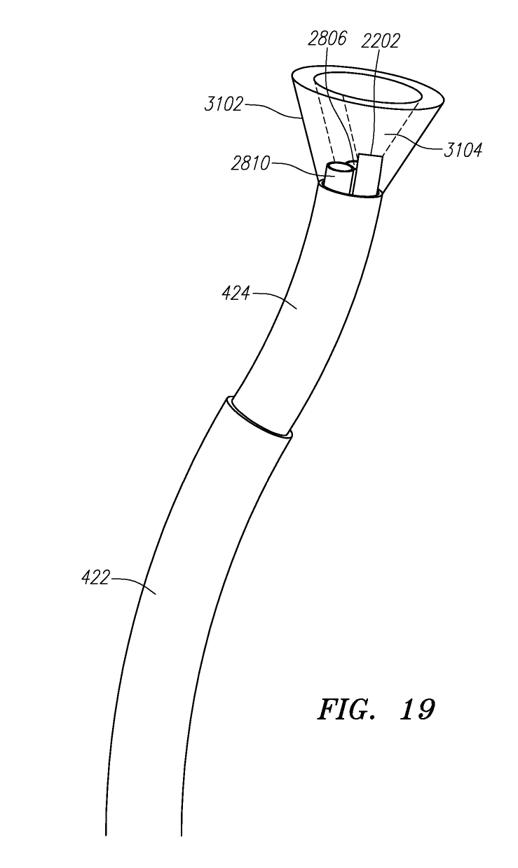

FIG. 6 depicts a similar configuration as the instrument assembly (108) including the sheath (422) and guide (424) that is illustrated in FIG. 17. FIG. 18 illustrates a basket tool (18026) and energy source (17036), such as a lithotripsy fiber (16026), positioned through the working lumen of the guide instrument (424). In each of the configurations depicted in FIG. 17 and FIG. 18, the energy source (17036) may be coupled to the pertinent capture device, or may be independently positioned through the working lumen of the guide instrument (424) to the desired location adjacent the capture device (17026, 18026). Each of the tools described herein, such as graspers, baskets, and energy sources, may be controlled proximally as they exit the proximal end of the working lumen defined by the guide instrument (424), or they may be actuated manually, automatically or electromechanically, for example through the use of electric motors and/or mechanical advantage devices. For example, in one embodiment, a configuration such as that depicted in FIG. 18, the sheath (422) and guide (424) instruments are preferably electromechanically operated utilizing an instrument driver (106) (not shown in these two figures) such as that described in the aforementioned patent application (11/481,433). The grasping mechanisms (17026, 18026) may be manually actuated, for example utilizing a positioning rod and tension wire, or electromechanically operated using a servomechanism or other proximal actuation devices. The energy source (17036) may be operated proximally utilizing a switch, such as a foot pedal or console switch, which is associated with the proximal energy control device (not shown in FIGS. 5 and 6).

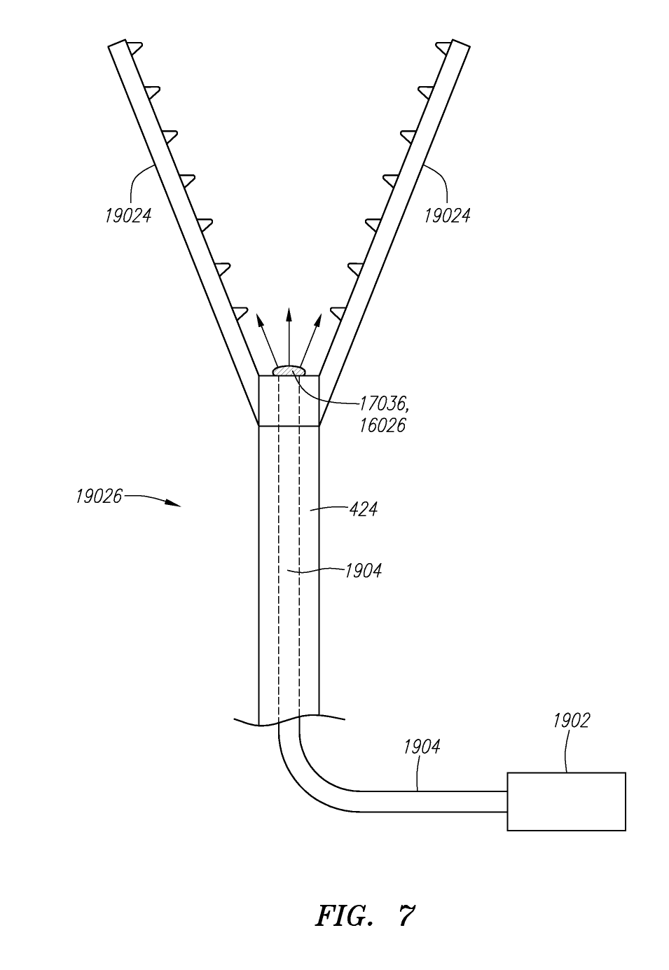

FIG. 7 depicts an expandable grasping tool assembly (19026) with an energy source (17036, 16026) mounted at the apex of the grasper mechanism. The energy source (17036, 16026) is proximally associated, by one or more transmission leads (1904), such as a fiber or wire, with a device (1902) such as an RF generator or laser energy source. The opposing jaws (19024) of the depicted grasping tool assembly (19026) are biased to spring outward, thus opening the grasper when unbiased. When pulled proximally into a confining structure, such as a lumen of a guide instrument (424), the hoop stress applied by the confining structure urges the jaws (19024) together, creating a powerful grasping action.

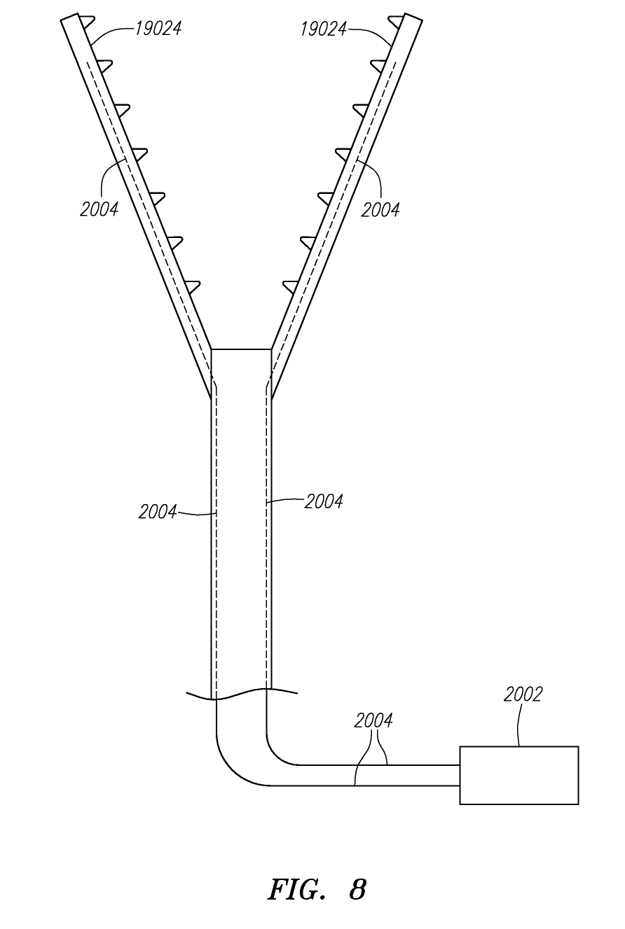

FIG. 8 depicts a bipolar electrode grasper with a proximally associated RF generator or other energy source (2002). In this embodiment, each of the jaws (19024) is biased to swing outward, as in the embodiment depicted in FIG. 19, and each of the jaws (19024) also serves as an electrode for the bipolar pairing, to be able to apply energy to items or objects which may be grasped. Leads (2004) are depicted to couple the jaws (19024) with a proximally positioned energy source (2002), such as an RF generator

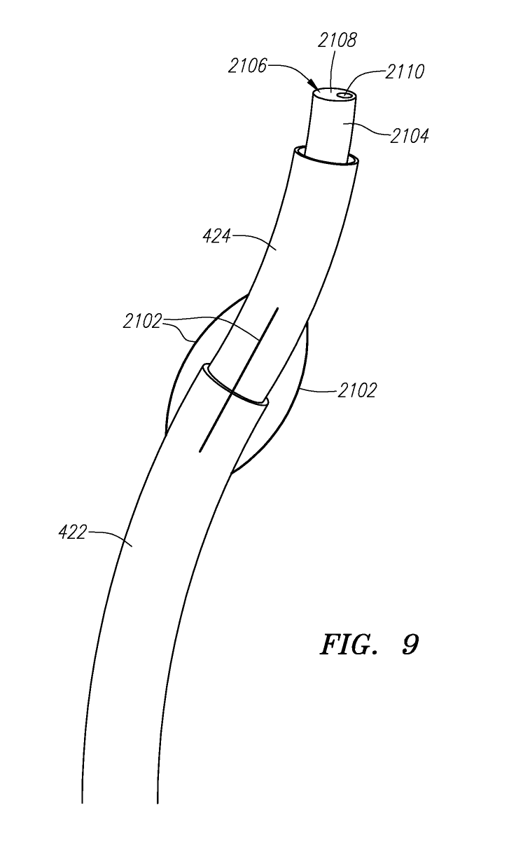

FIG. 9 depicts a sheath instrument (422) coupled to a group of basket arms (2102) that are biased to bend inward (i.e., toward the longitudinal axis of the sheath/guide as depicted), and configured to grasp a stone or other object as the guide instrument (424) is withdrawn proximally into the sheath instrument (422). The depicted embodiment features an image capture device (2104) which may or may not have a lens (2106), illumination fibers (2108) to radiate light, infrared radiation, or other radiation, and a working lumen (2110) for positioning tools distally. The image capture device (2104), which may comprise a fiberscope, CCD chip, infrared imaging device, such as those available from CardioOptics Incorporated, ultrasound device, or other image capture device, may be used, for example, to search for objects such as stones, and when located, the guide instrument (424) may be withdrawn into the sheath instrument (422) to capture the object, which the entire assembly is gently advanced to ensure that the object remains close to the distal tip of the assembly for easy capture by the basket device (2102)

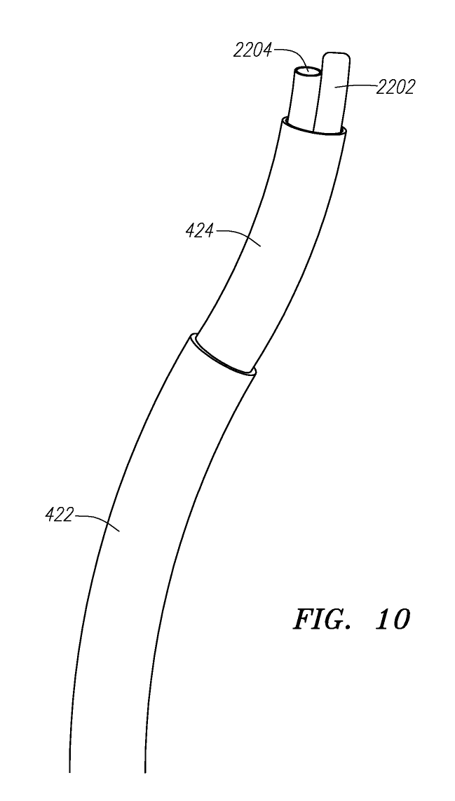

FIG. 10 depicts an assembly comprising a lithotripsy fiber (2202) and image capture device (2204) configured to enable the operator to see and direct the laser fiber (2202) to targeted structures, utilizing, for example, the high-precision navigability of the subject sheath (422) and guide (424) instrument assembly (108), and apply energy such as laser energy to destroy or break up such structures. Preferably the image capture device (2204) is positioned to include the position at which the energy source (such as a lithotripsy fiber 2202) as part of the field of view of the image capture device (2204)—i.e., to ensure that the operator can utilized the field of view to attempt to bring the energy source into contact with the desired structures.

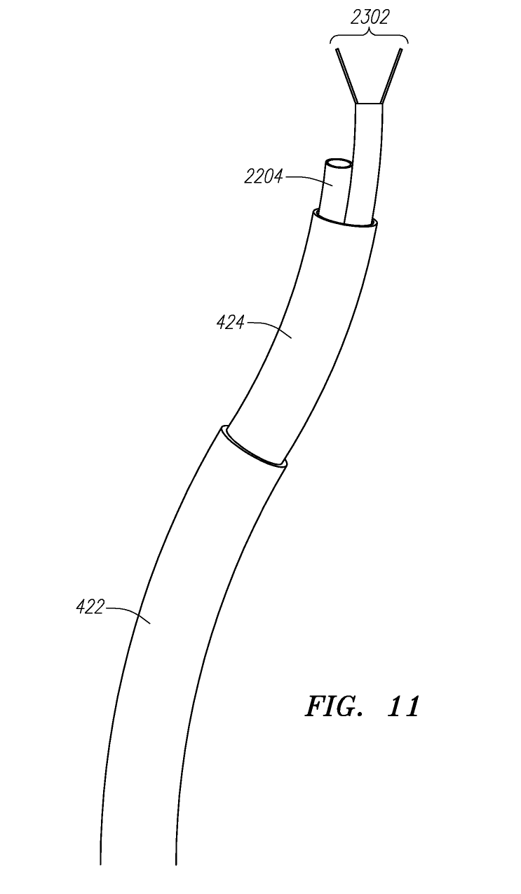

FIG. 11 depicts a similar embodiment as the one shown in FIG. 10, which includes a grasping tool (2302) to grasp a stone or other object and bring it proximally toward the image capture device (2204), such that it may be examined, removed proximally through the working lumen of the guide instrument (424), etc.

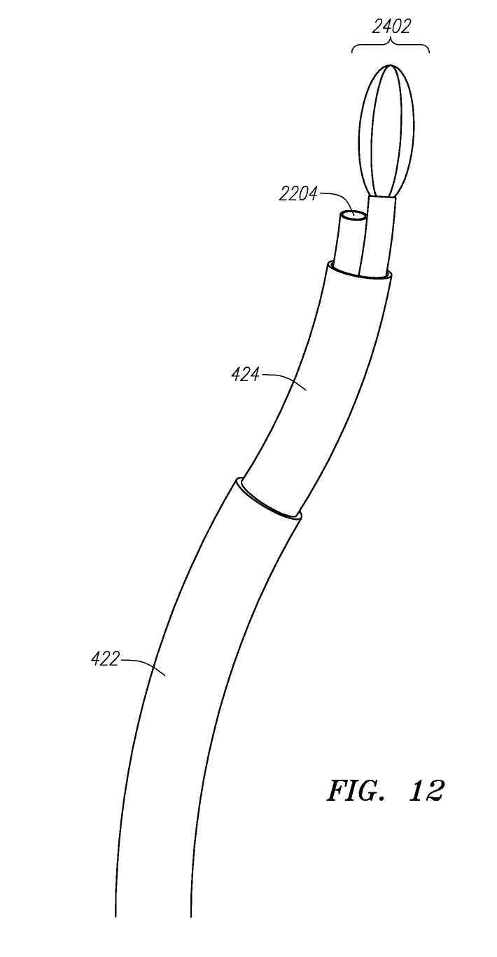

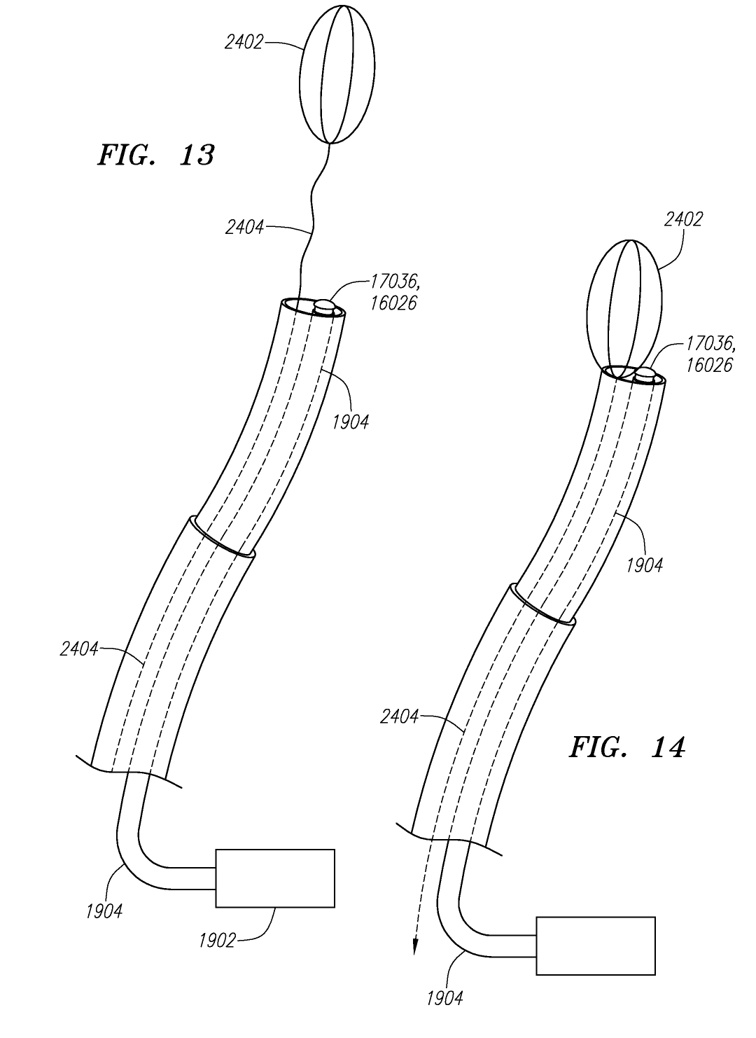

FIG. 12 illustrates another similar embodiment, which includes a basket tool (2402). FIG. 13 and FIG. 14, illustrate how an embodiment such as one depicted in FIG. 12 may be used to grasp and retrieve stones or other objects toward the distal portion of the guide (424). As the retrieved object approaches the guide (424), energy source (17036, 16026) breaks up the object in the basket tool (2402).

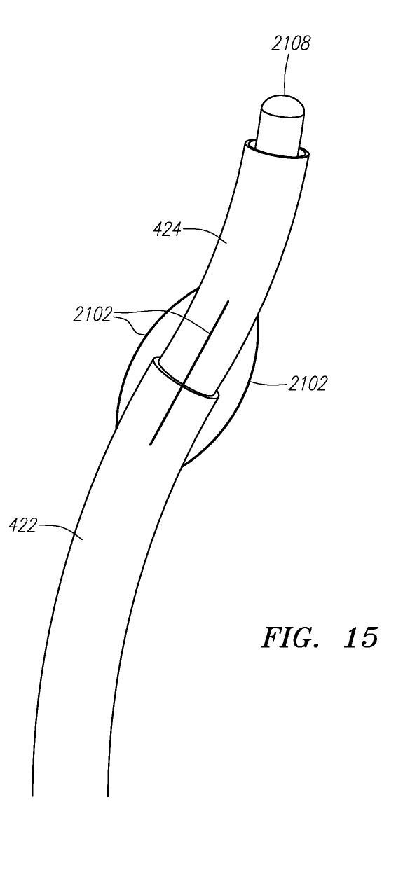

FIG. 15 depicts an embodiment with a proximal basket arm capture (2102) and an image capture device (2108). As described above in the portion of the description describing FIG. 9, when an object is observed with the image capture device (2108), the entire assembly may be advanced while the guide instrument (424) is withdrawn proximally into the sheath instrument (422) until the depicted basket capture arms (2102) are able to rotate toward the central axis of the guide instrument (424) working lumen and capture objects positioned adjacent the distal tip of the guide instrument (424)

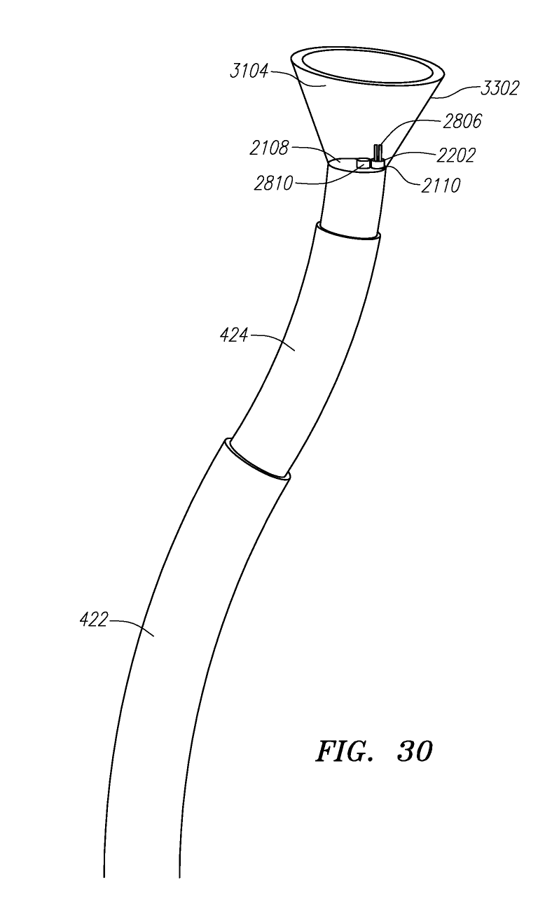

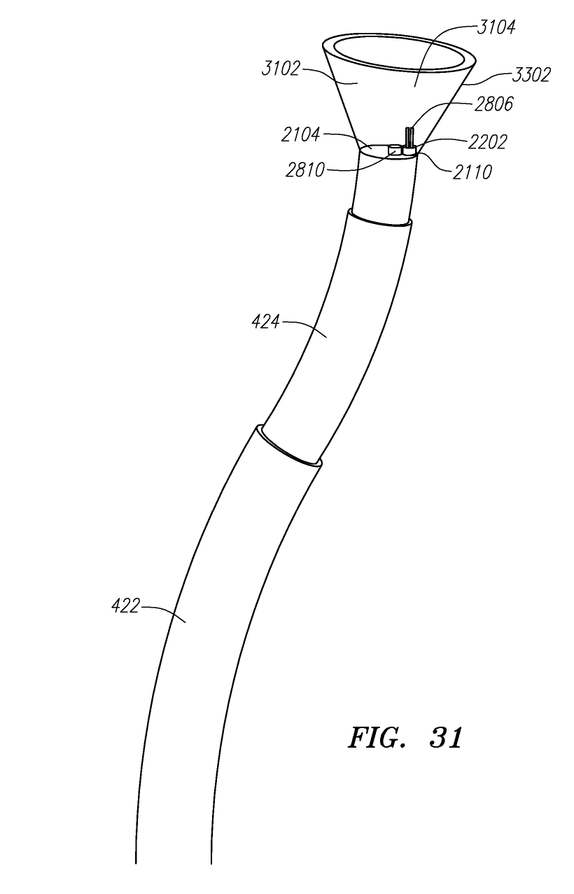

FIG. 16 depicts a configuration with an inflatable balloon (2802) configured to be controllably filled with or evacuated of saline (2804), through which an image capture device (2204) and illumination source (2806) may be utilized to observe objects forward of the balloon that preferably fall within the field of broadcast (2808) of the illumination source (2806) and field of view (2810) of the image capture device (2204). The balloon (2802) also defines a working lumen (2812) through which various tools may be passed—such as a laser fiber (2202), as depicted. FIG. 17 depicts a similar embodiment also comprising a grasping tool (2302). FIG. 30 depicts a similar embodiment with a basket tool (2402).

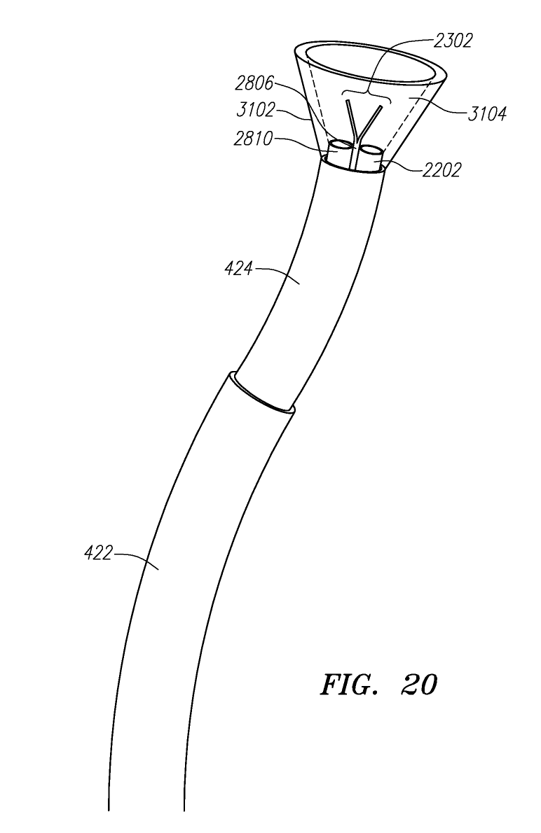

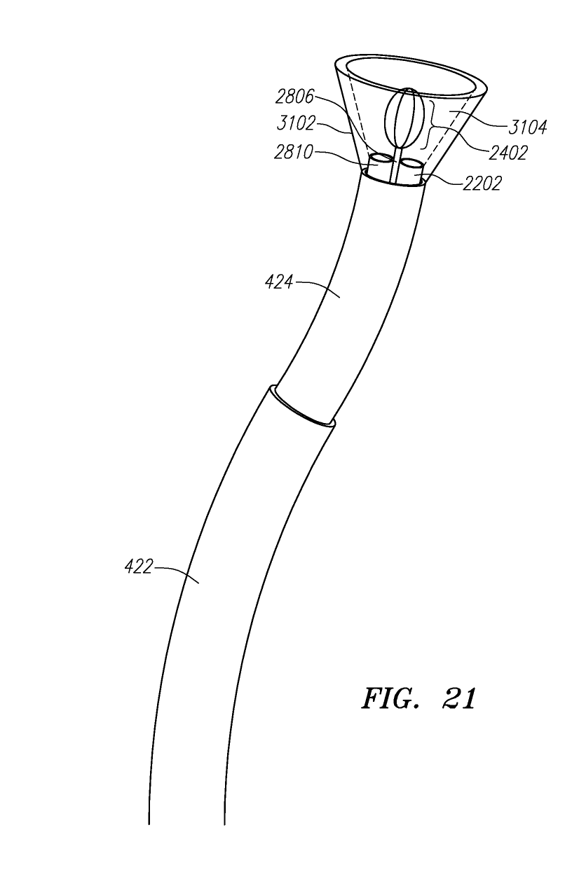

FIG. 19 through FIG. 21 depict similar embodiments which comprise an inflatable balloon cuff (3102) configured to provide a distal working volume (3104) which may be flushed with a saline flush port (2806). The inflatable balloon cuff (3102) preferably works not only as an atraumatic tip, but also as a means for keeping the image capture device (2810) positioned slightly proximally of structures that the inflatable balloon cuff (3102) may find itself against—thus providing a small amount of volume to image such structures without being immediately adjacent to them. With an optical fiberscope as an image capture device (2810), it may be highly valuable to maintain a translucent saline-flushed working volume (3104) through which the image capture device (2810) may be utilized to image the activity of objects, such as tissues and/or kidney stones, as well as the relative positioning of tools, such as fibers, graspers, baskets, etc., from proximal positions into the working volume (3104)—which may be used, for example, to grasp and/or modify or destroy stones or other structures. The inflatable balloon cuff (3102) may be advanced to the desired operational theater, such as the calices of a kidney, in an uninflated configuration, and then inflated in situ to provide the above functionality. Alternatively, the cuff (3102) may be inflated before completing the navigation to the operational theater, to provide atraumatic tip functionality as well as image capture guidance and deflection from adjacent objects, during navigation to the desired operational theater.

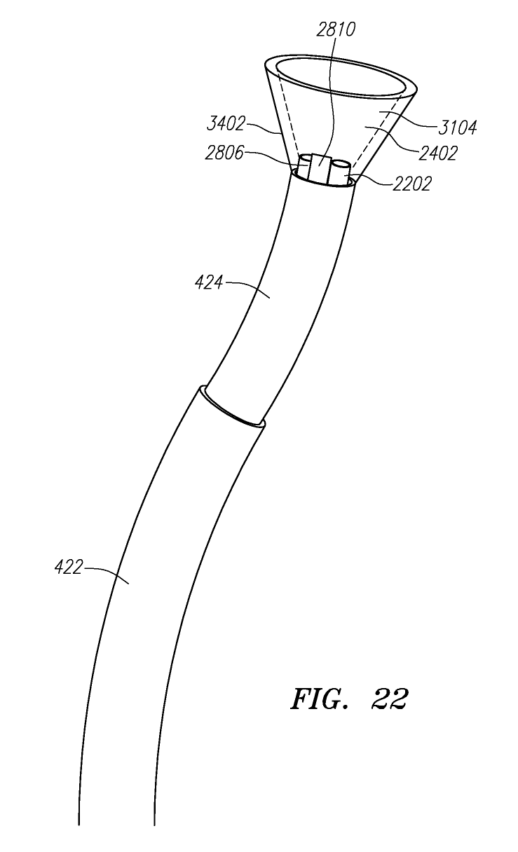

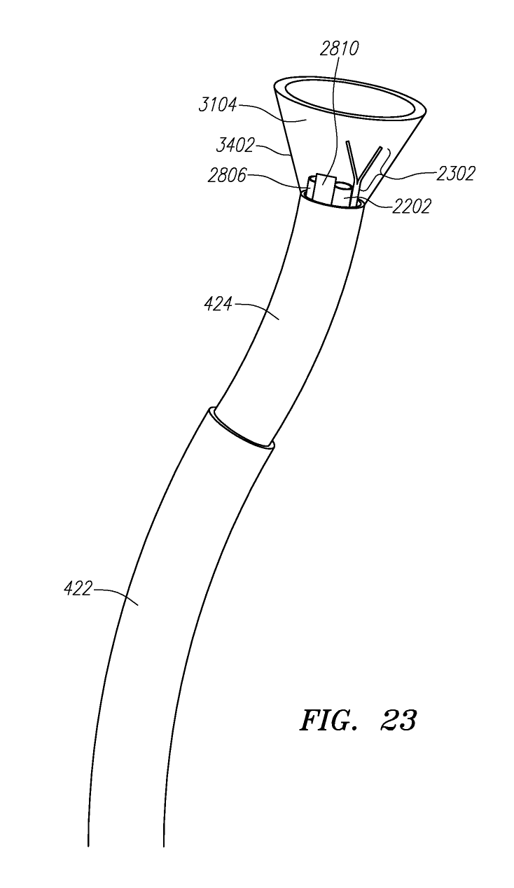

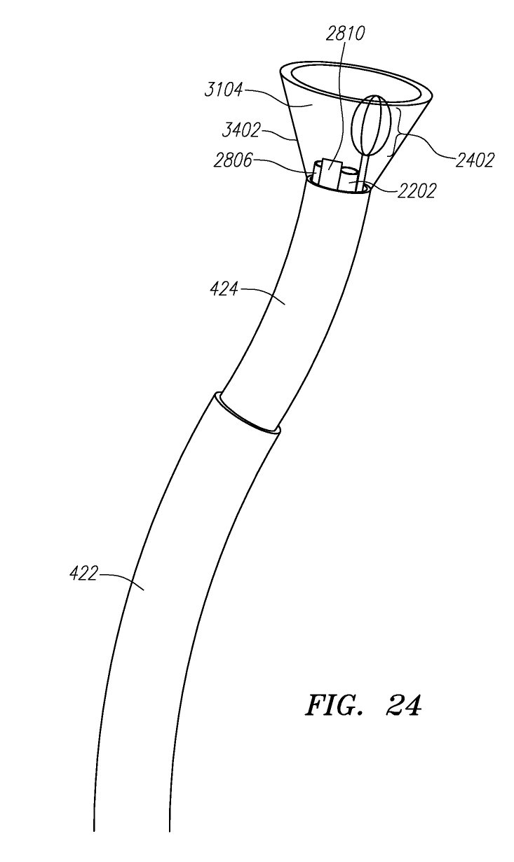

FIG. 22 through FIG. 24 depict similar embodiments, but with a flexible cuff (3402), preferably comprising a soft polymer material, rather than an inflatable cuff (3102) as in the previous set of figures. The flexible cuff (3402) is configured to have similar functionalities as those described in reference to the inflatable cuff (3102) above.

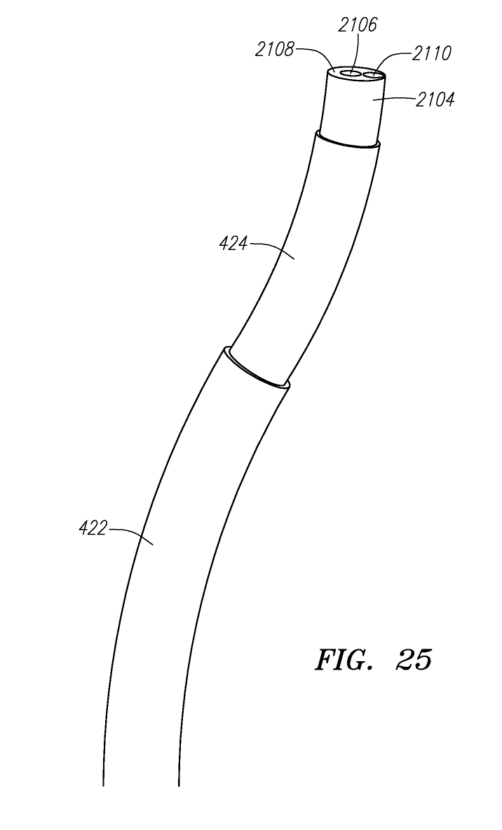

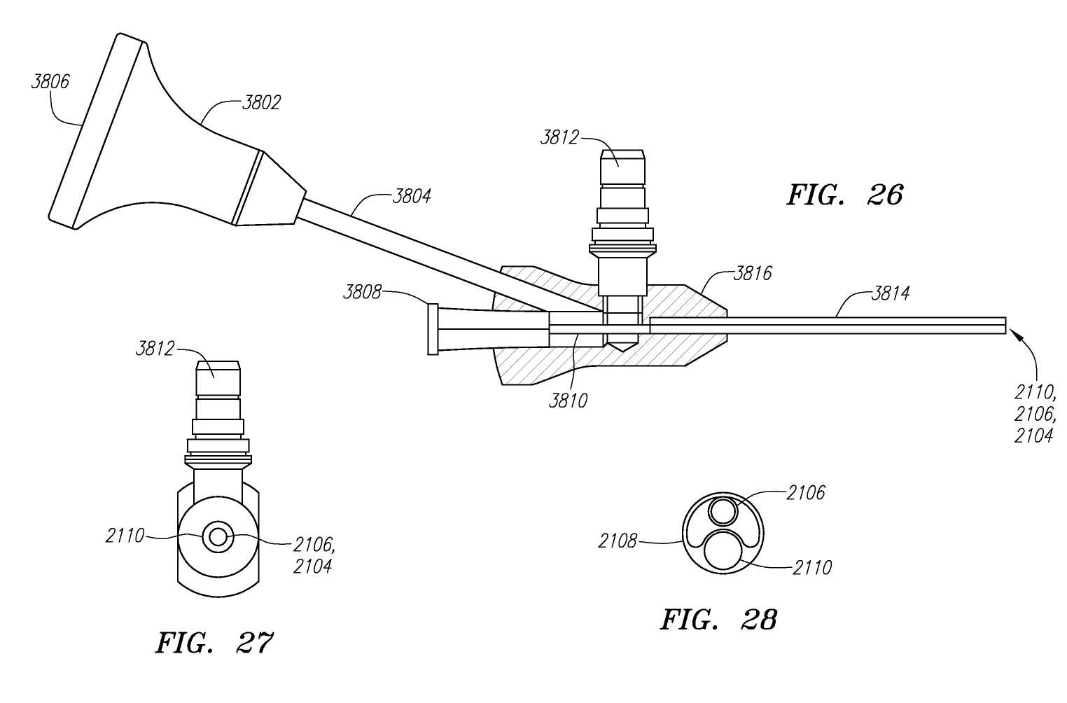

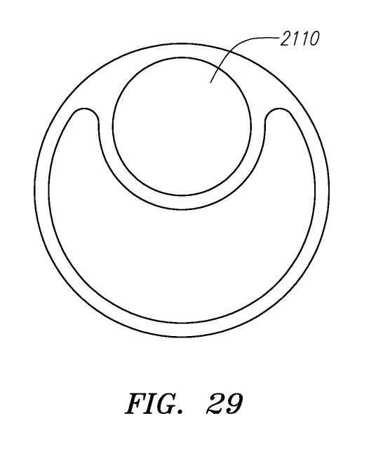

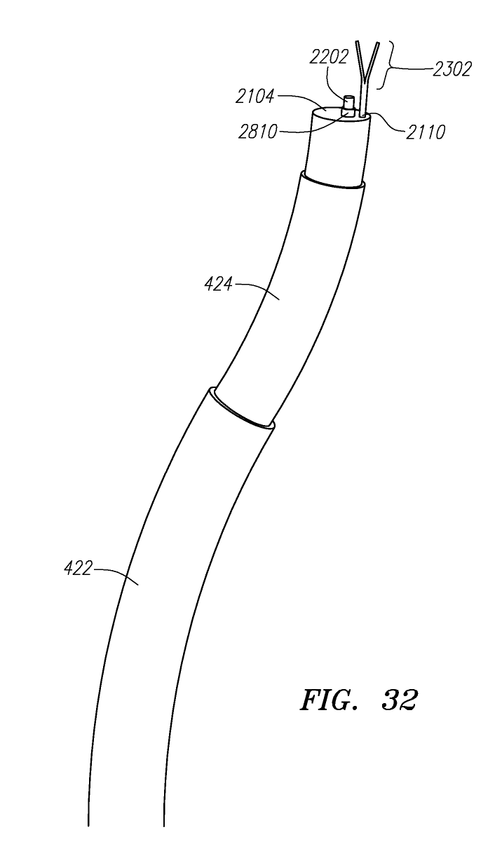

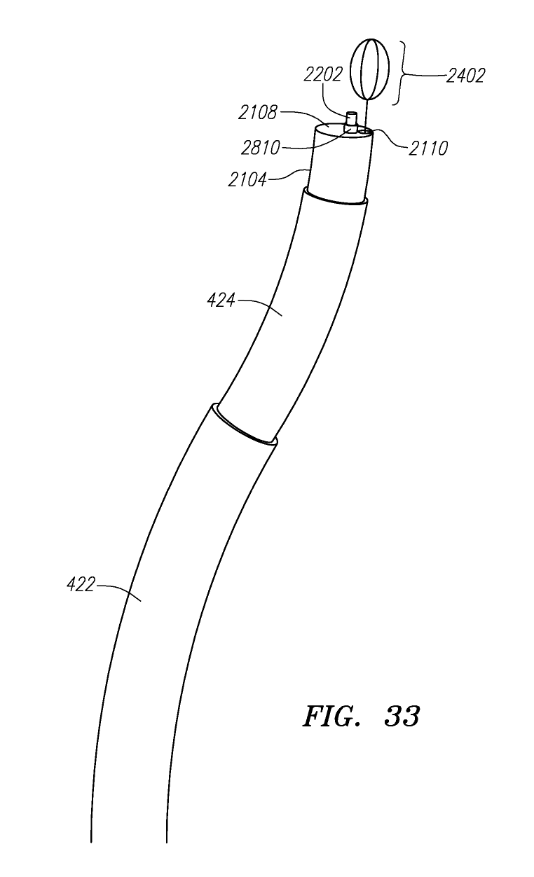

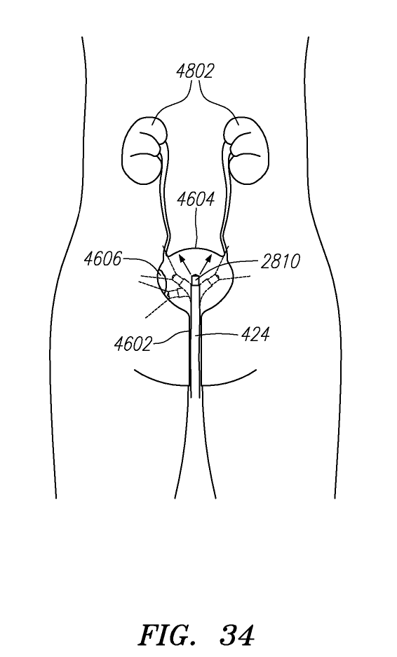

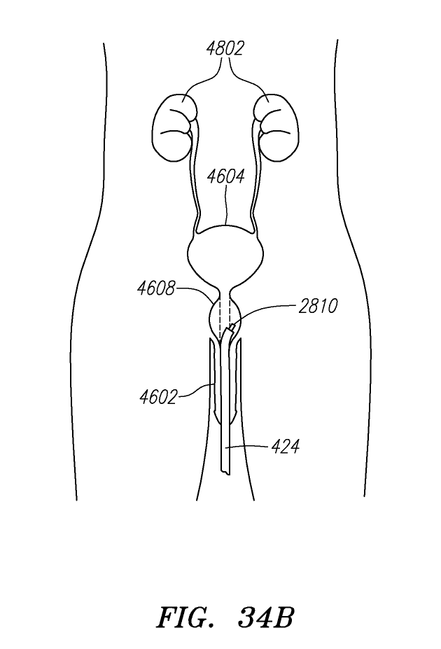

FIG. 25 through FIG. 29 depict an embodiment wherein an assembly of an image capture device (2104), which may optionally comprise a lens (2106), transmission fibers (2108) for imaging, and a working lumen (2110), through which various tools or combinations of tools may be positioned. The components of this embodiment are all packaged within one tubular structure as illustrated in the cross sectional view of FIG. 29, which may comprise a co-extruded polymeric construct. FIG. 26 through FIG. 28 depict the interconnectivity of an image capture device (2104), such as a fiberscope comprising a proximal optics fitting (3802), an optics body member (3804), a proximal surface (3806) for interfacing with a camera device with the illumination fibers and working lumen, comprising a female luer fitting (3808) for accessing the working lumen (2110), a working lumen proximal member (3810), an illumination input tower (3812), an insertion portion (3814), a central body structure (3816). Variations of this embodiment are depicted in FIG. 30 through FIG. 33, with different distal configurations similar to those depicted in reference to the figures described above. FIG. 30 depicts a variation having a distally-disposed flexible cuff (3402) defining a working volume (3104) flushable with a saline port (2806) and imaged with an image capture device (2810) as described above. FIG. 31 depicts a similar variation having an inflatable cuff (3102). Tools such as graspers, energy sources, fibers, baskets, etc may be utilized through the working lumens (2110) of the embodiments depicted in FIG. 30, FIG. 31, FIG. 32, FIG. 33, etc. The embodiment of FIG. 34 comprises a grasping tool (2302) positioned through the working lumen of the assembly (2104—the assembly depicted in FIG. 25 through FIG. 29), which the embodiment of FIG. 33 comprises a basket tool (2402).

Each of the above discussed tools, configurations, and/or assemblies may be utilized for, among other things, endolumenal urinary intervention, such as the examination, removal, fragmentation, and/or destruction of stones such as kidney or bladder stones.

Referring to FIG. 34, a steerable instrument assembly according to one embodiment may be steered through the urethra (4602) and into the bladder (4604), where an image capture device (2810) may be utilized, as facilitated by injected saline, to conduct a cystoscopy and potentially observe lesions (4606) of interest. The omni-directional steerability and precision of the robotic guide and/or sheath to which the image capture device is coupled facilitates collection of images of inside of the bladder (4606) which may be patched together to form a 3-dimensional image. The instrument assembly (108-422, 424, 2810) may also be utilized to advance toward and zoom the image capture device upon any defects, such as obvious bleeds or tissue irregularities. Similar procedures may be performed in the prostate (4608) as illustrated in FIG. 34B.

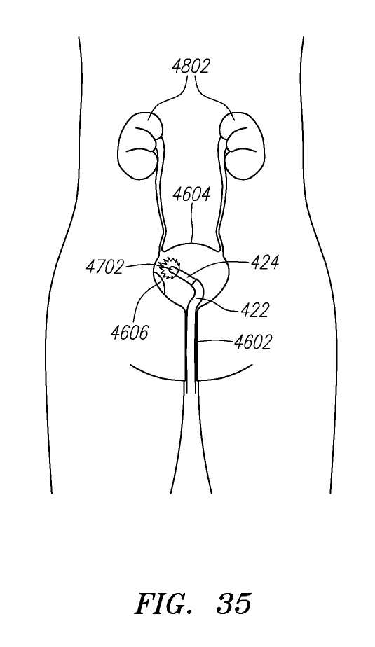

Referring to FIG. 35, the instrument assembly (108-422, 424, 4702) may alternatively or additionally include an interventional tool such as an ablation tool (4702) for ablating tumors or other lesions (4606) within the bladder (4604) or prostate. Any of the above-discussed assemblies may be utilized for such a cystoscopy procedure.

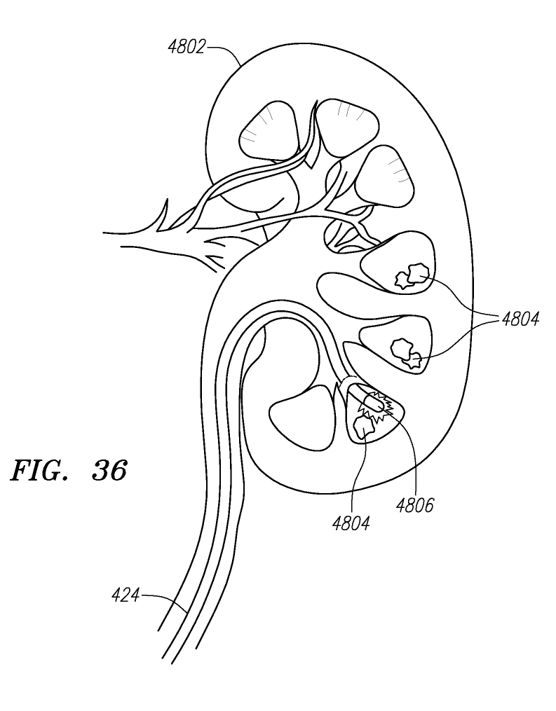

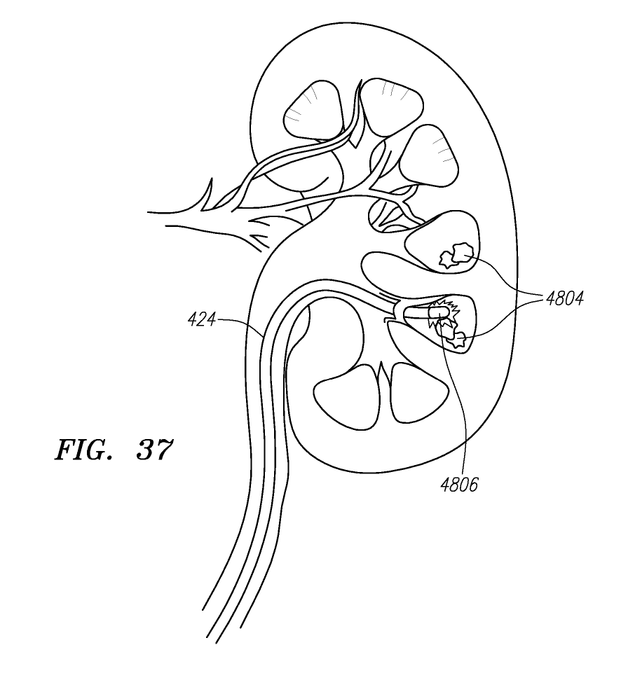

Each of the above-discussed constructs may also be utilized adjacent to or within the kidneys. Referring to FIG. 36 and FIG. 37, for illustrative purposes, a portion of a relatively simple instrument assembly embodiment (for example, a sheath distal tip may be positioned in the bladder at the entrance to the urethra while the more slender guide, 424, is driven toward and into the kidney, 4802) is depicted. Such assembly may be advanced toward and/or steerably driven into the kidney (4802), where stones (4804) may be captured with graspers or other tools, or where stones may be destroyed using chemistry, cryo, RF, laser lithotripsy, or laser ablation tools (4806), or other radiative techniques, such as ultrasound, as depicted in FIG. 36 and FIG. 37. Each of the tools, configurations, and/or assemblies discussed above in reference to FIG. 4 through FIG. 33 may be utilized for the examination, removal, fragmentation, and/or destruction of stones such as kidney or bladder stones. Preferably, an image capture device (2810) is positioned in or adjacent to the calices of the kidney to enable interactive viewing of objects such as stones, while various tool configurations may be utilized to examine, capture, grasp, crush, remove, destroy, etc, such stones, before withdrawing the instrument assembly.

All of the aforementioned balloons, cuffs, ablation tools, electrodes, etc. apparatuses are configured to be operatively coupled to the instrument assembly (108) in combination with the sheath catheter (422) and guide catheter (424). In some embodiments, the tools or instruments, e.g., balloons, ablation tools, electrodes, etc., may be used with the guide catheter (424) without the sheath catheter (422). In other embodiments, additional catheters may be used with the tools or instruments. As apparent to one skilled in the art, the tools and instruments are configured to be either manually operated or robotically operated by the instrument driver (106) in connection with the instrument (108). Some of the circuitry, electrical, and mechanical systems for controlling and operating all of the aforementioned tools and instruments may be configured at the instrument driver (106) and the system electronics rack (114).

While multiple embodiments and variations of the many aspects of the invention have been disclosed and described herein, such disclosure is provided for purposes of illustration only. Many combinations and permutations of the disclosed system, apparatus, and methods are useful in minimally invasive medical diagnosis and intervention, and the invention is configured to be flexible and adaptable. The foregoing illustrated and described embodiments of the invention are suitable for various modifications and alternative forms, and it should be understood that the invention generally, as well as the specific embodiments described herein, are not limited to the particular forms or methods disclosed, but also cover all modifications, alternatives, and equivalents as defined by the scope of the appended claims. Further, the various features and aspects of the illustrated embodiments may be incorporated into other embodiments, even if not so described herein, as will be apparent to those skilled in the art. In addition, although the description describes data being mapped to a three dimensional model, data may be mapped to any mapping or coordinate system, including two dimensional, static or dynamic time-varying map, coordinate system, model, image, etc. All directional references (e.g., upper, lower, upward, downward, left, right, leftward, rightward, top, bottom, above, below, vertical, horizontal, clockwise, counterclockwise, etc.) are only used for identification purposes to aid the reader's understanding of the invention without introducing limitations as to the position, orientation, or applications of the invention. Joining references (e.g., attached, coupled, connected, and the like) are to be construed broadly and may include intermediate members between a connection of elements (e.g., physically, electrically, optically as by an optically fiber, and/or wirelessly connected) and relative physical movements, electrical signals, optical signals, and/or wireless signals transmitted between elements. Accordingly, joining references do not necessarily infer that two elements are directly connected in fixed relation to each other. It is intended that all matters contained in the description or shown in the accompanying drawings shall be interpreted as illustrative only and not limiting. Modifications, alternatives, and equivalents in the details, structures, or methodologies may be made without departing from the spirit and scope of the invention as defined by the appended claims.

What is claimed is:

1. A robotic surgical system, comprising:
an instrument driver;
an operator control station operatively coupled to the instrument driver via a remote communication link, wherein user inputs received at the operator control station control movements of mechanisms in the instrument driver;
an instrument assembly operatively coupled to the instrument driver such that the mechanisms of the instrument driver operate or control movements of components of the instrument assembly, the instrument assembly including a flexible elongate guide instrument and a component instrument carried in a lumen of the guide instrument, the component instrument comprising a light source, an image capture device, and an optical tissue treatment fiber.

2. The robotic surgical system of claim 1, the instrument assembly further including an elongate sheath instrument, wherein the guide instrument is carried in, and movable relative to, the sheath instrument.

3. The robotic surgical system of claim 1, wherein the optical tissue treatment fiber comprises a laser fiber.

4. The robotic surgical system of claim 1, wherein the component instrument further comprises a grasper.

5. The robotic surgical system of claim 4, wherein the grasper is movable relative to the guide instrument.

6. The robotic surgical system of claim 1, wherein the component instrument further comprises a basket apparatus.

7. The robotic surgical system of claim 6, wherein the basket apparatus is movable relative to the guide instrument.

8. The robotic surgical system of claim 1, wherein the component instrument further comprises an inflatable balloon carried on a distal end thereof.

9. The robotic surgical system of claim 8, wherein the light source and image capture device are located in an interior of the balloon.

10. The robotic surgical system of claim 1, wherein the component instrument further comprises a cuff apparatus carried on a distal end thereof.

11. The robotic surgical system of claim 8, wherein the light source and image capture device are located in an interior of the cuff apparatus.

12. The robotic surgical system of claim 3, wherein the laser fiber is a lithotripsy laser fiber.

13. The robotic surgical system of claim 12, wherein the lithotripsy laser fiber is a Holmium YAG laser.
